(19) United States
(12) Patent Application Publication
(10) Pub. No.: US 2024/0298896 A1
(43) Pub. Date: Sep. 12, 2024

(54) VARIABLE DEPTH TRANSILLUMINATION DEVICE

(71) Applicant: Nizar Mullani, Sugar Land, TX (US)

(72) Inventor: Nizar Mullani, Sugar Land, TX (US)

(21) Appl. No.: 18/424,014

(22) Filed: Jan. 26, 2024

Related U.S. Application Data
(60) Provisional application No. 63/488,913, filed on Mar. 7, 2023.

(51) Int. Cl. A61B 5/00

(52) U.S. Cl. CPC A61B 5/0059; A61B 5/489

(57) ABSTRACT

A variable depth transillumination device has an orange light source, with LEDs having a wavelength in the range of 580-620 nm, and a red light source with LEDs having a wavelength of light in the range of approximately 620-660 nm, and, if desired, a yellow light source having a wavelength in the range of 560 nm to 580 nm. The device utilizes a user-controlled variable control switch that enables a user to vary the intensity of angled colored LEDs to facilitate variable depth viewing of veins in one device. Orange light-emitting LED sources are beneficial for transillumination of superficial veins and red light-emitting LED sources are beneficial for transillumination of deeper veins. Yellow light-emitting LEDs are beneficial for transillumination of even more superficial veins. A combination of the two colored LEDs enables a user to view a variety of depths shallow and deep with one device.

### CROSS-REFERENCE TO RELATED APPLICATIONS

The application claims the benefit of U.S. Patent Application Ser. No. 63/488,913 filed Mar. 7, 2023.

### BACKGROUND OF THE INVENTION

The invention relates to a variable depth transillumination device. More particularly, the invention relates to a variable depth transillumination device having an angled orange LED (light emitting diode) light source and a different angled red LED light source.

Transillumination entails the shining of a light through a body cavity or organ for diagnostic purposes. Typically, transillumination is performed in a room where the lights have been dimmed or turned off to facilitate the viewing of the part being studied. A bright light is pointed at the cavity or organ and, due to the slight translucence of the part under consideration, some of the light passes through the part or organ. This test is often performed on newborns or infants with hydrocephalus or males suspected of having hydrocele. In addition, transillumination is used for tests performed on breast tissue to detect lesions and/or cysts. In newborns, the test is used to transilluminate the chest cavity if pneumothorax is suspected. Only in newborns is transillumination of the chest possible. Transillumination is painless and quickly performed with inexpensive equipment. It is often the only method of finding veins in prematurely born babies (neonates).

Transilluminators use color to facilitate the viewing of the tissue organ under study. U.S. Pat. No. 4,651,743, issued to Stoller, discloses a transillumination device using red light. In addition, U.S. Pat. No. 5,957,917, issued to Doiron et al., discloses that red light is particularly useful for performing transillumination of tissue for diagnostic purposes. Further, U.S. Pat. No. 7,874,698, issued to Mullani, relates to a transillumination device having an orange color light. These devices use colored light at a specified wavelength to illuminate veins that are positioned below a skin surface. Typically, the vein transilluminator uses a side-transillumination method where the light shines into the skin from the top and at a single angle to the skin so that the light is focused approximately 2 cm to 4 cm below the skin, based on the wavelength and angle of the light. In some devices, a plurality of light sources is positioned in a circular configuration defining an annulus of focused light. The annulus of focused light behaves like a virtual light source under the skin and transilluminates an area of the skin inside the circle of light.

In the past, multiple devices were used for the evaluation of various depths of transillumination. For example, when it is the desire for the user to examine deeply below the skin of a patient, a separate device that uses specific red LED lights of a particular wavelength and angling is used by the medical practitioner. In order to do an exam of veins or other items just below the skin of a patient, a separate orange LED light source of a particular wavelength is used by the medical practitioner. As a result, various types of devices were required for complete transillumination; ones for transillumination of veins or items that are not too deep below the surface of the skin, and ones for examination of veins or items that have a deeper location. In some cases, a single device had two colors of LEDs, orange and red, in one device, but none of these transillumination devices allow for varying depths to be examined by a single device.

### SUMMARY OF THE INVENTION

In one aspect, a variable depth transillumination device includes, in combination, a housing having a first end, a second end, a front surface, and a bottom surface. An angled orange first light source, and an angled red second light source where both arrays are located at a first end portion of a housing and along the front surface of the housing. The first light source is composed of an array of angled orange LEDs producing light having a wavelength in the range of 580-620 nm (preferably 590 nm) for illumination of shallow veins. The second light source is composed of an array of angled red LEDs producing light having a wavelength in the range of 620-660 nm (preferably 660 nm) for transillumination of deeper veins. The angle of the orange LEDs of the first light source is different from the angle of the red LEDs of the second light source. Each of the LEDs is angled to facilitate the illumination of a specific location and depth beneath the surface of the skin of a patient. Notably, the orange LEDs of the first light source are angled at approximately 30 to 60 degrees relative to the planar surface defined by the back surface of the housing and the red LEDs of the second light source are angled at approximately 70 to 90 degrees relative to the planar surface defined by the back surface of the housing. The transillumination device further includes a variable power source and a variable light control switch for adjusting the amount of transillumination produced by each array of LEDs in relation to one another. The variable power source and variable light control switch allow for proportionate adjustment of each array of LEDs, orange and red, to facilitate illumination of a specific depth below the surface of the skin of a patient. As the power is adjusted between arrays of LEDs the intensity of the light of each array changes in relation to one another. For example, the intensity of the red light will increase, and the intensity of the orange light will decrease.

It is also an object of the invention to provide a unique portable, battery-powered transillumination device that combines separate and distinct functions, namely, transillumination of shallow veins and transillumination of deep veins, as well as transillumination of all depths therebetween as a result of an adjustment of the variable power source and switch. The variable power source and variable light control switch control the amount of power sent to each array of LEDs. Therefore, different functions employ separate and distinct light sources, and the variation of the power source transmission to each array of LEDs, red and orange, enables viewing of all depths, shallow and deep, based on the variation of power to each array of LEDs. The variable light control switch enables light emitted by the red emitting LEDs to move in inverse intensity to light emitted by the orange emitting LEDs.

It is another object of the invention to provide a transillumination device with two arrays of angled LEDs, one angled array of red LEDs and one angled array of orange LEDs. The red LEDs are angled at approximately 80 degrees (from 70 to 90 degrees) relative to the planar surface defined by the back surface and the orange LEDs are angled at approximately 45 degrees (from 30 to 60 degrees) relative to the planar surface defined by the back surface of the housing. The transillumination device can easily switch between the two types of LED light sources, or a combination of the two colors simultaneously, in various power illuminations, to create a very measured light source for examination of veins and other items at all various depths below a skin's surface with one device.

These and other objects of the invention will become apparent to one of ordinary skill in the art after reviewing the disclosure of the invention.

### BRIEF DESCRIPTION OF THE DRAWINGS

FIG. 1 is a front side view of the transillumination device.

FIG. 2 is a bottom side view of the transillumination device.

FIG. 3 is a perspective view of the transillumination device.

FIG. 4 is a front side view of an alternate embodiment of the circular-shaped ring of a transillumination device.

FIGS. 5 and 6 are perspective views of the transillumination device in use.

### DETAILED DESCRIPTION OF THE INVENTION

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

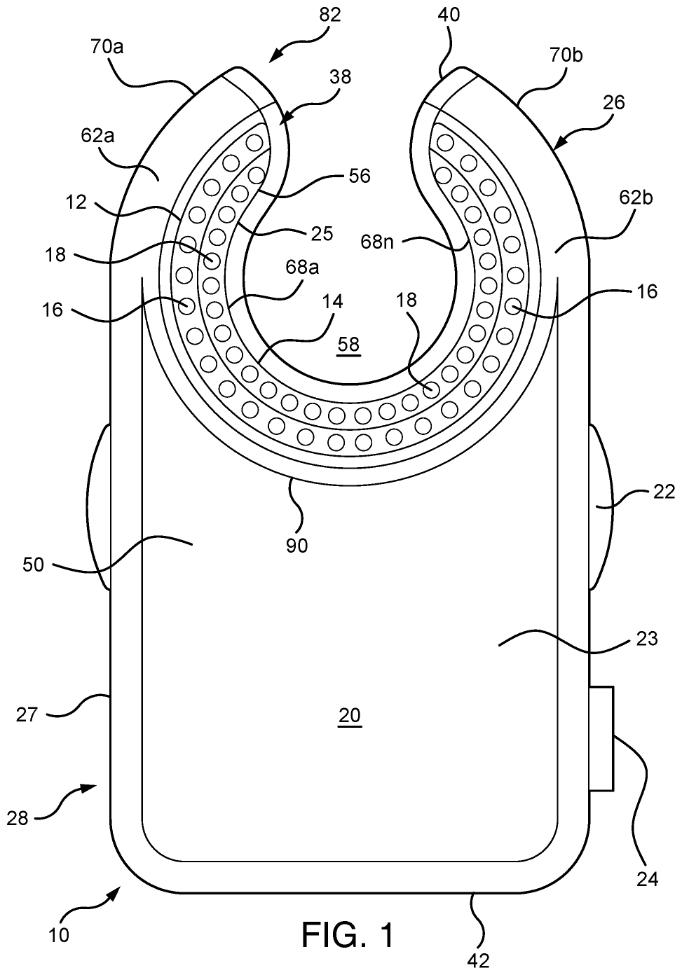

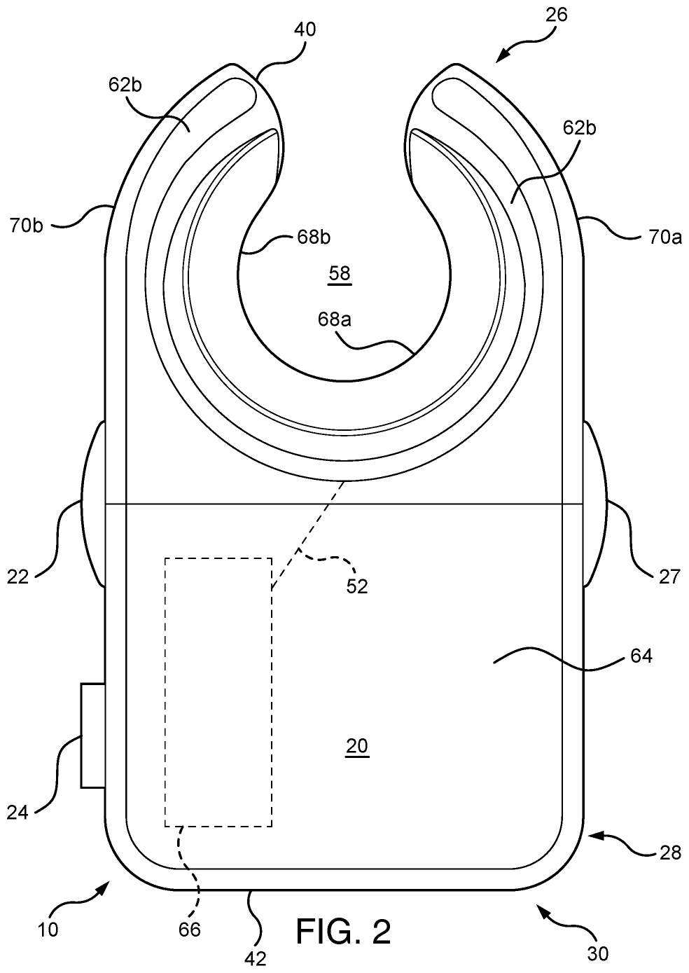

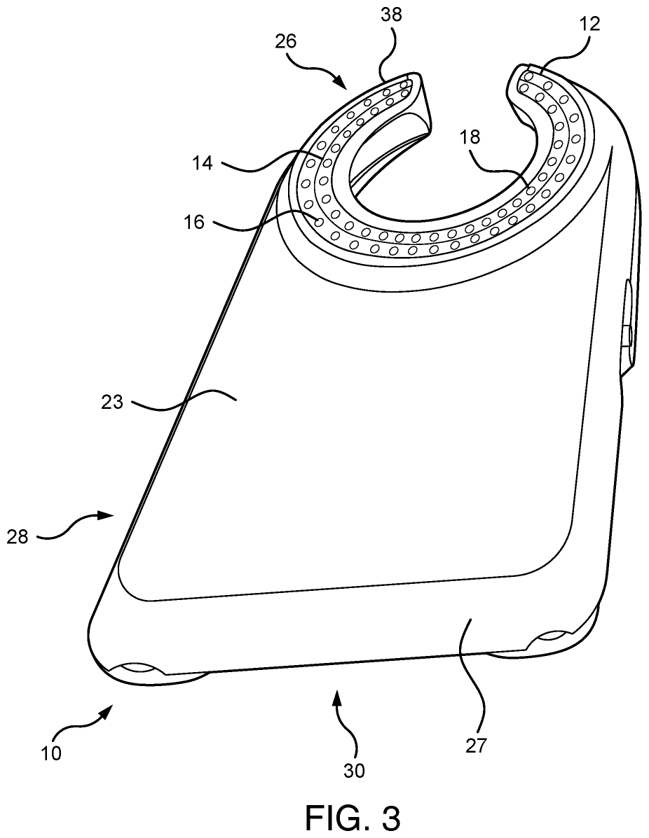

Referring to FIGS. 1 to 3, 5, and 6 a transillumination device 10 of the present invention has an orange first light source 12 composed of a first array of LEDs emitting orange light 16 having a wavelength in the range of 580-620 nm and a red second light source 14 composed of a second array of LEDs emitting red light 18 having a wavelength of light in the range of 620-660 nm. Orange light has been found to be beneficial for transillumination of superficial veins and red light has been found to be beneficial for transillumination of deeper veins. Further, orange LED light has higher absorption in venous blood than red LED light and penetrates deeper in darker, pigmented skin; thus, allowing better vein transillumination in dark-skinned patients. A combination of both orange light and red light allows for all depths therebetween to be viewed.

In addition to the first array of LEDs emitting orange light 16 and the second array of LEDs emitting red light 18 projecting light of a different color (wavelength), the first array of LEDs emitting orange light 16 and the second array of LEDs emitting red light 18 project light at different angles to the skin. The combination of different wavelengths and angles allows for different depth examinations.

It should also be appreciated that there is no requirement that the number of red LEDs is the same as the number of orange LEDs in an embodiment. Since red LED light penetrates deeper than orange LED light, there are situations where providing more orange LED lights will be required.

The transillumination device 10 has a portable configuration and includes an on/off switch (or switching mechanism) 22 that allows the user to easily switch the transillumination device 10 on and off. The transillumination device 10 also includes a user-controlled variable light control switch 24 enabling current to be varied between the orange first light source 12 and the red second light source 14 by simply turning a switch (for example, a toggle switch) that toggles between two contact points, one contact point for shallow veins and one contact point for deeper veins. By toggling between the contact points, the light source being activated is selected and the depth of view is varied. While a toggle switch is disclosed herein, it is appreciated the switch could be a slider switch with the same features ranging between shallow and deep. The user-controlled variable light control switch 24 can also be a variable resistor switch. By adjusting the amount of power delivered to the orange first light source 12 and the red second light source 14, one may selectively vary the depth of a patient's veins to be viewed. In addition, the transillumination device 10 has a battery compartment (not shown) that houses a battery to facilitate its portable usage.

In accordance with a disclosed embodiment, when the on/off switch 22 is first depressed the first array of LEDs emitting orange light 16 and/or the second array of LEDs emitting red light 18 project light for side transillumination of veins. The amount of orange light and/or red light emitted by the first array of LEDs emitting orange light 16 and/or the second array of LEDs emitting red light 18 is dependent upon the user-controlled variable light control switch 24. The user then achieves transillumination by placing the front surface 50 of the housing 20 directly upon the skin. In particular, the side-transillumination shines light into the skin from the top and at an angle to the skin so that the light is preferably focused approximately 2 to 4 cm below the skin. The annulus of focused light behaves like a virtual light source under the skin and transilluminates an area of the skin inside the circle of light.

Although the disclosed embodiment is manually controlled, it is contemplated an automatic sensor can be utilized such that when the unit is in touch with the skin, it will switch on the desired light source. In all instances for the device to properly function it must be in direct contact with the skin of a patient.

A power source 66 is associated with the user-controlled on/off switch 22 (for example, a button) used to turn the transillumination device 10 on and off. In accordance with some embodiments, the button that is used to turn the device on and off can also be programmed to turn on the orange LED lights first by a first push and then the red LED lights by a second push. This type of configuration allows for quick examination of veins that are shallow and deep. As explained above, the user-controlled variable light control switch 24 is provided for use in conjunction with the on/off switch 22 for adjusting and controlling power sent to each of the first and second light sources 12, 14, in a controlled manner for the selective examination of a user's shallow veins, deeper veins, or depths therebetween. The user-controlled variable light control switch 24, which sends power to one or both of the arrays of LEDs using a resistor mechanism, is positioned along the exterior surface of the housing 20 to allow for ready access by users of the transillumination device 10. The ability to switch colors from one switch or vary the amount of illumination from each of the colors is designed for maximum ease when working at various depths. The user-controlled variable light control switch 24 can be located on the bottom surface of the housing, or on a side of the housing 20 (as shown), enabling a user to switch between the projection of orange light, red light, or intensities of combinations therebetween for the desired illumination depth.

The transillumination device 10, as depicted in FIG. 1, includes a housing 20 having an elongated rectangular shape. The housing 20 includes a front side 23, a back side 64, and a plurality of side walls 27 extending between the front side 23 and the back side 64. The housing 20 further includes a first end portion 26 and a second end portion 28. As discussed below in greater detail, the first end portion 26 includes a circular-shaped illumination portion 38. The housing 20 is structured to be portable and small for ease of use. The housing 20 is elongated as it extends from a front edge 40 at the first end portion 26 to a rear edge 42 at the second end portion 28.

The first end portion 26 of the housing 20 has a substantially circular-shaped end when viewed along a plane as shown in FIGS. 1, 2 and 3. Although the housing 20 is overall generally shaped as a rectangle, the first end portion 26 of the housing 20 is circular-shaped. A front surface 50 defines the front side 23 of the housing 20 and extends between the front edge 40 and the rear edge 42 along the long axis of the housing 20. The housing also includes a back surface 52 that defines the back side 64 of the housing 20. As with the front surface 50, the back surface 52 extends between the front edge 40 and the rear edge 42 along the long axis of the housing 20. The back surface 52 is substantially flat and defines a planar surface.

While the housing 20, and ultimately the transillumination device 10, is described in accordance with a preferred embodiment as being substantially rectangular-shaped, it is appreciated that it may take a variety of shapes as the preferences of different users might dictate.

The housing 20 of the transillumination device 10 includes a handle portion 30 at the second end portion 28 thereof and an illumination portion 38 with the first array of LEDs emitting orange light 16 (that is, the orange first light source 12) and the second array of LEDs emitting red light 18 (that is, the red second light source 14) at the first end portion 26. The illumination portion 38 defines a circular-shaped ring, which contains the first array of LEDs emitting orange light 16 and the second array of LEDs emitting red light 18.

Referring to the handle portion 30, it is substantially rectangular-shaped, although it is appreciated that it may take a variety of shapes as the preferences of different users might dictate. Regardless of its shape, the handle portion 30 is shaped and dimensioned to allow a user to securely grip the handle portion.

The housing 20 contains the power source 66 for the first array of LEDs emitting orange light 16 and the second array of LEDs emitting red light 18. In accordance with a preferred embodiment, the power source 66 is substantially contained within the handle portion 30 and is associated with the on/off switch 22 and user-controlled variable light control switch 24 allowing for actuation of the first array of LEDs emitting orange light 16 and the second array of LEDs emitting red light 18 in a controlled manner for the selective examination of a user's skin or veins.

As for the illumination portion 38 of the housing 20, it includes a substantially circular-shaped (for example, horseshoe-shaped) area of illumination. The illumination portion 38 may be thought of as including a proximal end 90 that merges into the handle portion 30 and a distal end 82 at the front edge 40 of the housing 20. The circular-shaped area of illumination is oriented such that it extends from the front edge 40 of the housing 20 at a distal end 82 of the illumination portion 38 to the proximal end 90 adjacent the handle portion so as to form a viewing area.

More particularly, the circular-shaped area of illumination is defined by first and second arcuate arms 62a, 62b at the first end portion 26 of the housing 20. Each of the first and second arcuate arms 62a, 62b include a concave inner surface 68a, 68b and a convex outer surface 70a, 70b wherein the concave inner surfaces 68a, 68b face each other so as to form a central opening 58 in the housing 20 at the first end portion 26 through which a user looks as he or she is viewing the transilluminated body portion over which the transillumination device 10 is positioned. The first and second arcuate arms 62a, 62b extend to enclose a space of approximately 270 degrees. Each of the arms 62a, 62b tapers inwardly along the concave inner surfaces 68a, 68b as they extend from the back surface 52 of the housing 20 to the front surface 50 of the housing 20, focusing a user's eye upon the center of the opening 58 when the user looks downwardly upon the back surface 52 of the housing 20 toward light being transmitted away from the front surface 50 of the housing 20.

A circular recess 25 is formed in the front surface 50 of the housing 20 in the area of the first and second arcuate arms 62a, 62b at the first end portion 26 of the housing 20. The first array of LEDs emitting orange light 16 and the second array of LEDs emitting red light 18 are positioned within the circular recess 25 so as to project light away from the front surface 50 and toward a body portion as will be discussed below in greater detail. The recess 25 includes a substantially circular central space 56 in which the first array of LEDs emitting orange light 16 and the second array of LEDs emitting red light 18 are positioned.

As briefly discussed above, the first array of LEDs emitting orange light 16 and the second array of LEDs emitting red light 18 housed within the recess 25 project light from the front surface 50 of the housing 20 for illumination of the skin and/or transillumination of a vein as desired; that is, the orange first light source 12 and the red second light source 14 are formed in the front surface 50 of the housing 20 in the illumination portion 38. The orange first light source 12 and the red second light source 14 are shaped and dimensioned to encircle the substantially circular central space of the opening 58. Accordingly, the illumination portion 38 is of a semicircular shape extending about an arc of approximately 270 degrees as defined by first and second arcuate arms 62a, 62b. The orange first light source 12 and the red second light source 14 are focused to project light of differing predetermined frequencies toward the center of the substantially circular central space.

The recess 25 may be thought of as defining a substantially circular (or oval-shaped) projection area from which the light generated by the first and second light sources 12, 14 is projected. In accordance with a preferred embodiment, the transillumination device 10 includes a transparent glass or plastic lens 70 secured along the back surface 52 of the housing 20 so as to cover the recess 25 in which the first and second light sources 12, 14 are positioned. The transparent glass or plastic lens 70 is selected so as to allow for the complete passage of the light generated by the first and second light sources 12, 14 therethrough for projection into the skin of a patient being studied.

As discussed above, the illumination portion 38 is composed of the orange first light source 12 and the red second light source 14, which are respectively composed of a plurality of LEDs. The LEDs of the orange first light source 12 are a first array of LEDs emitting orange light 16. The LEDs are angled to facilitate a specific depth viewing. In accordance with a preferred embodiment, the first array of LEDs emitting orange light 16 produce orange light with a wavelength between 580 and 620 nm. The use of orange light with a wavelength between 580 and 620 nm allows for enhanced imaging of superficial veins. The orange LEDs are angled at approximately 30 to 60 degrees, more specifically 45 degrees, relative to the planar surface defined by the back surface 52 of the housing and ultimately a surface to be viewed. The red second light source 14 includes a plurality of LEDs generating red light; that is, the second array of LEDs emitting red light 18. The LEDs are angled to facilitate a specific depth viewing. In accordance with a preferred embodiment, the second array of LEDs emitting red light 18 produces light with frequencies between 620 and 650 nm enabling the viewing of deeper veins through darker skin. The red LEDs are angled at approximately 70 to 90 degrees, more specifically 80 degrees, relative to the planar surface defined by the back surface 52 of the housing 20 and ultimately a surface to be viewed.

It is appreciated the transillumination device 10 may have different color light sources in one ring that can transmit both orange and/or red LED light which enables side transillumination of veins of different depths, as desired. Orange light has higher absorption in venous blood than red light whereas red light penetrates deeper in darker, pigmented skin, thus allowing better vein illumination in dark-skinned patients. It is appreciated the angular orientation of the red and orange LEDs is defined above relative to the back surface 52 of housing 20 and the back surface might not be flat. As such, it would be necessary to define a different point of reference as those skilled in the art would certainly appreciate.

In accordance with a disclosed embodiment as shown with reference to FIGS. 1 to 3, the first and second light sources 12, 14 are located in separate rings positioned within the recess 25 and extend along the substantially circular-shaped area of illumination so as to project red and/or orange light toward a patient, at a specific angular orientation, being examined. The pattern of the first and second light sources 12, 14 extends about the substantially circular-shaped area of illumination and begins at a position of approximately 10 o'clock as shown in FIG. 1, and extends in a counterclockwise direction to a position of approximately 1 o'clock.

As discussed above, each of the first and second light sources 12, 14 is composed of distinct LEDs used to create different light colors as needed by the medical practitioner using the transillumination device 10. The orange first light source 12 is composed of a plurality of LEDs generating orange light and the second light source 14 is composed of a plurality of LEDs generating red light. The LEDs of the first and second light sources 12, 14 are all contained within the housing 20 and the light shines through the lens 70. This combination of light sources enables both deep and shallow vein transillumination.

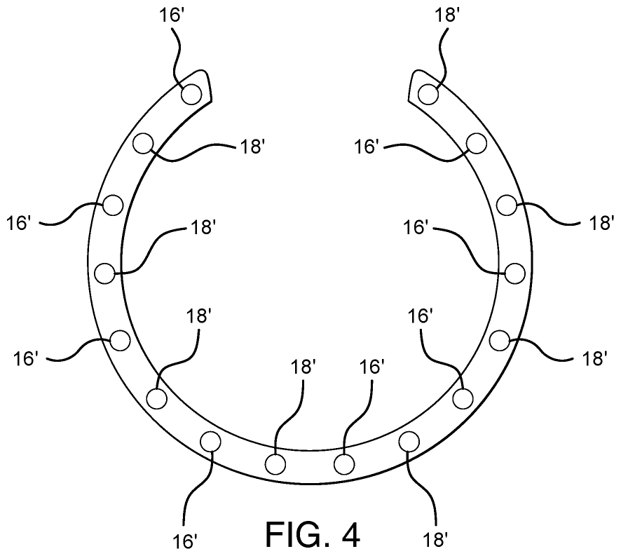

While FIGS. 1 to 3 disclose an embodiment where the LEDs are placed in separate rings, one for each color LED, it is appreciated the LEDs can be all located in one ring in an alternating fashion as shown with reference to FIG. 4. The angle of the color of each LED, orange or red, remains at the precise angle range discussed above to facilitate various depth illumination. In particular, the illumination portion 38 contains a single ring of orange LEDs defining the first array of LEDs emitting orange light 16 and red LEDs defining the second array of LEDs emitting red light 18. The LEDs alternate between red and orange. This enables the circular-shaped ring to be smaller and can be used for small children and infants.

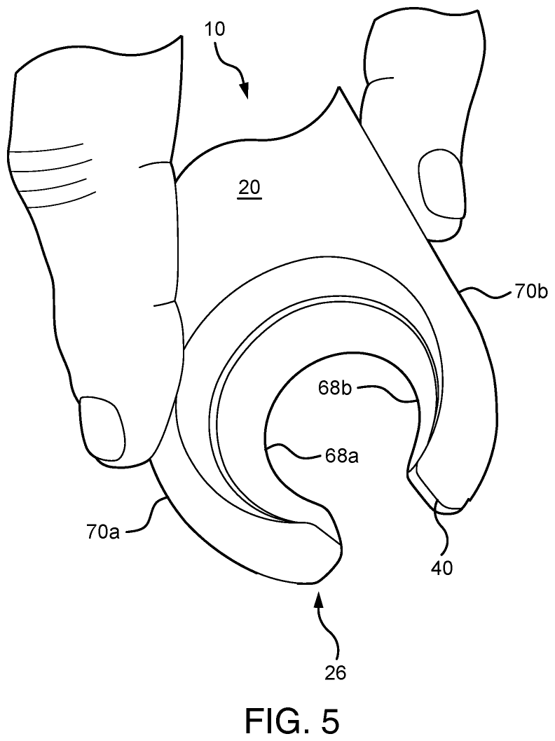

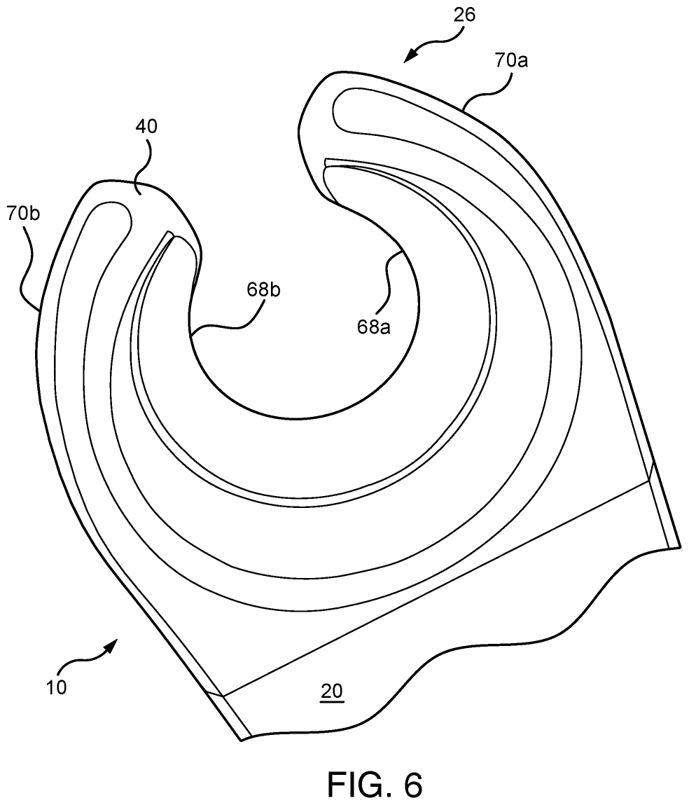

In practice, and with reference to FIGS. 5 and 6, the transillumination device 10 is placed directly onto the patient's skin for vein or skin surface imaging, as desired. Other applications of transillumination devices are well known. In use, and after the transillumination device 10 has been turned on when the user-controlled variable light control switch 24 is first engaged, light from the orange first light source 12 is illuminated projecting either orange light from the series of LEDs for transillumination of shallow veins or red light for the transillumination of deeper veins. When the user-controlled variable light control switch 24 is adjusted, less orange LED light is produced, and some red LED light is then produced enabling a user to change the viewing depth of the projected light. When the user-controlled variable light control switch 24 is adjusted in the direction of the red LED setting less orange light is projected and the viewing of a deeper location is enabled. The combination of LED intensities can also be incrementally adjusted for use to enable different depths to be viewed at the same time. The front surface 50 of the housing 20 of the transillumination device 10 is preferably positioned in contact with the skin allowing the selected LED lights to illuminate the vein or location of a location in the skin of a patient for examination. In this way, the transillumination device 10 enables the user to view an almost infinite selection of depths of a vein or item to be viewed using a single portable device. The ability to have one device to view shallow and deep veins and all locations therebetween provide significant savings to the user, by enabling such to occur in one simple handheld device.

There is also provided a method of viewing both shallow and deep veins of a patient by using the compact transillumination device. The method includes the steps of using the transillumination device as described above to perform a variable depth examination without the examiner having to use a separate device and interrupt the examination. Further still, the examination is done via body transillumination, and side-transillumination is not required. Never before has a device capable of variable depth imaging of arterial blood vessels been provided.

The embodiments disclosed above include an orange first light source composed of a plurality of LEDs generating orange light and a red second light source composed of a plurality of LEDs generating red light. It is contemplated the range of light colors emitted by the transillumination device could be expanded with the addition of a third light source, for example, a yellow light source composed of a plurality of LEDs generating yellow light with a wavelength in the range of from 560 nm to 580 nm. As with the embodiments disclosed above, a user-controlled variable light control switch would be used to control all three of the light sources such that a desired light is transmitted toward the patient's skin. Yellow light-emitting LEDs have been found to be beneficial for transillumination of superficial veins. The third set of yellow light-emitting LEDs can be provided in a separate ring or interspersed between the red and orange LEDs,

While the invention has been described with reference to a preferred embodiment, variations and modifications would be apparent to one of ordinary skill in the art. Such variations and modifications are encompassed by the invention.

What is claimed is:

1. A variable depth transillumination device comprising a housing, a circular-shaped ring of light emitting diodes retained by the housing for vein imaging, the circular-shaped ring of light emitting diodes comprising an outer ring of orange light emitting LEDs, the orange light emitting LEDs angled at from 30 to 60 degrees From a surface to be viewed and an inner ring of red light emitting LEDs, the red light emitting LEDs angled at from 70 to 90 degrees From the surface to be viewed.

2. The variable depth transillumination device as claimed in claim 1, wherein an intensity of the red light emitting LEDs and an intensity of the orange light emitting LEDs is controlled by a variable light control switch, that enables light emitted by the red light emitting LEDs to move in inverse intensity to light emitted by the orange LEDs.

3. The variable depth transillumination device as claimed in claim 1, wherein the circular-shaped ring of light emitting diodes comprises the orange light emitting LEDs and red light emitting LEDs in a staggered formation between the inner ring and the outer ring.

4. A variable depth transillumination device comprising a housing, a circular-shaped ring of light emitting diodes retained by the housing for vein imaging, the circular-shaped ring of light emitting diodes comprising orange light emitting LEDs, the orange light emitting LEDs are angled from 30 to 60 degrees and red light emitting LEDs, the red light emitting LEDs angled at from 70 to 90 degrees, wherein the orange light emitting LEDs and the red light emitting LEDs alternate around the circular-shaped ring.

5. The variable depth transillumination device as claimed in claim 1, wherein the outer ring of orange light emitting LEDs are angled 45 degrees From the surface to be viewed and the inner ring of red light emitting LEDs are angled 80 degrees From the surface to be viewed.

6. The variable depth transillumination device as claimed in claim 1, wherein there are more orange LEDs than red LEDs.
